(19) United States
(12) Patent Application Publication
(10) Pub. No.: US 2008/0268403 A1
(43) Pub. Date: Oct. 30, 2008

(54) DENTAL TOWEL

(76) Inventor: Phillip Phung-I Ho, Santa Barbara, CA (US)

Correspondence Address: PAI PATENT & TRADEMARK LAW FIRM, 1001 FOURTH AVENUE, SUITE 3200, SEATTLE, WA, 98154, US

(21) Appl. No.: 11/739,693

(22) Filed: Apr. 24, 2007

(51) Int. Cl. A61C 19/00; A61F 13/15

(52) U.S. Cl. 433/137; 604/390

(57) ABSTRACT

A dental towel has at least four edges, a straight tear line and a surrounding band. One of the edges is oriented edge that is oriented forward a patient. The straight tear line is parallels the oriented edge, is near the oriented edge and has two ends and a length shorter than a length of the oriented edge. One of the ends is formed on an adjacent edge that is adjacent to the oriented edge. The surrounding band is mounted between the straight tear line and the oriented edge, surrounds conveniently a patient's neck and has an adhesive member sticking on the dental towel. The dental towel can be torn to obtain the surrounding band with a desired length to fit patient's neck. Thus, the dental towel can be positioned in front of patient's chest to prevent water or other agents from spraying to contaminate patient's clothes and neck.

### BACKGROUND OF THE INVENTION

1. Field of Invention

The present invention relates to a dental towel, and more particularly to a dental towel that is convenient for medical personnel to use and makes patients comfortable.

2. Description of the Related Art

A dental towel is used to cover a patient's chest to prevent water or other agents from spraying to stain the patient's clothes. Moreover, medical personnel are convenient to use the dental towel to clean patient's face.

A traditional dental towel is rectangular to cover a patient's chest, has four edges and is used with a binding line. The binding line has two ends. Each end has a clip to clip the dental towel to allow the line and the dental towel to surround patient's neck. However, the traditional dental towel must be used with the binding line with the clips, so it is inconvenient for medical personnel to obtain the line to position the dental towel in front of patient's chest.

With reference to FIG. 8, a conventional dental towel (20) is rectangular and has two opposite short edges (22) and two opposite long edges (21). One of the long edges (21) is cut from one of the short edges (22) to form a surrounding band (25). The surrounding band (25) has a specific length to surround a patient's neck and further has an end and an adhesive member (26). The adhesive member (26) is mounted on the end of the surrounding band (25) to paste on the dental towel (20) after the surrounding band (25) surrounding the patient's neck. Thus, it is convenient for medical personnel to use. However, the patient may be an adult or a child, a male or a female, whose necks have different dimensions, but the surrounding band (25) has only a specific length. Therefore, the surrounding band (25) may be too long for the patient whose neck with a small dimension, so the dental towel (20) is easy to move and can not be positioned in front of patient's chest and water or other agents will spray to stain patient's clothes and neck.

To overcome the shortcomings, the present invention provides a dental towel to mitigate or obviate the aforementioned.

### SUMMARY OF THE INVENTION

The primary objective of the present invention is to provide a dental towel that is convenient for medical personnel to use and makes patients comfortable.

To achieve the objective, the dental towel in accordance with the present invention has at least four edges, a straight tear line and a surrounding band. One of the edges is an oriented edge that is oriented forward a patient. The straight tear line parallels the oriented edge, is near the oriented edge and has two ends. A length is shorter than a length of the oriented edge. One of the ends formed on an adjacent edge adjacent to the oriented edge. The surrounding band is mounted between the straight tear line and the oriented edge, surrounds patient's neck and has an adhesive member. The adhesive member is mounted on the surrounding band to stick on the dental towel when the dental towel is used. The dental towel can be torn to obtain the surrounding band with a desired length to fit patient's neck. Thus, the dental towel can be positioned in front of patient's chest to prevent water or other agents from spraying to contaminate patient's clothes and neck.

Other objectives, advantages and novel features of the invention will become more apparent from the following detailed description when taken in conjunction with the accompanying drawings.

### BRIEF DESCRIPTION OF THE DRAWINGS

FIG. 1 is a front view of a dental towel in accordance with the present invention;

FIG. 2 is a front view of the dental towel in FIG. 1 with a straight tear line and a curved tear line being torn;

FIG. 3 is a front view of the dental towel in FIG. 2 with a removed part being removed and an adhesive member sticking on the dental towel;

FIG. 4 is a partially enlarged front view of the dental towel in FIG. 1 showing multiple triangular nodes;

FIG. 5 is a partially enlarged front view of the dental towel in FIG. 1 showing multiple rectangular nodes;

FIG. 6 is an operational perspective view of the dental towel in FIG. 1 covering an adult patient's chest;

FIG. 7 is an operational perspective view of the dental towel in FIG. 1 covering a minor patient's chest; and

FIG. 8 is a front view of a conventional dental towel in accordance with the prior art.

### DETAILED DESCRIPTION OF THE INVENTION

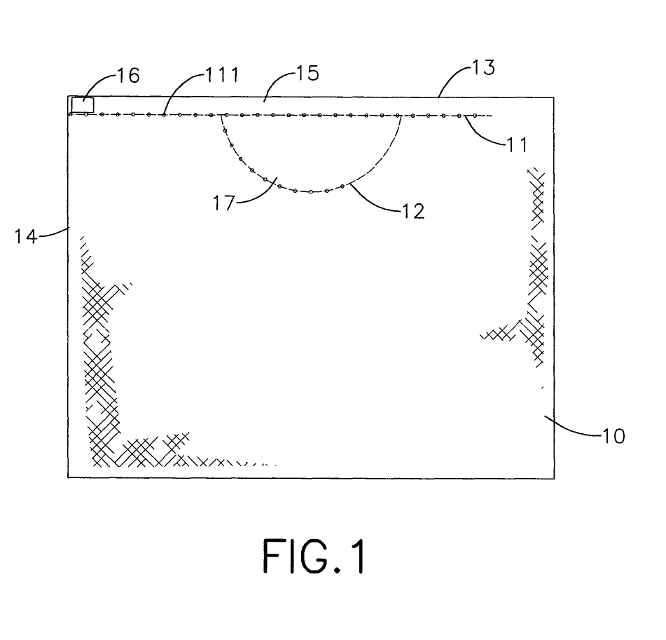

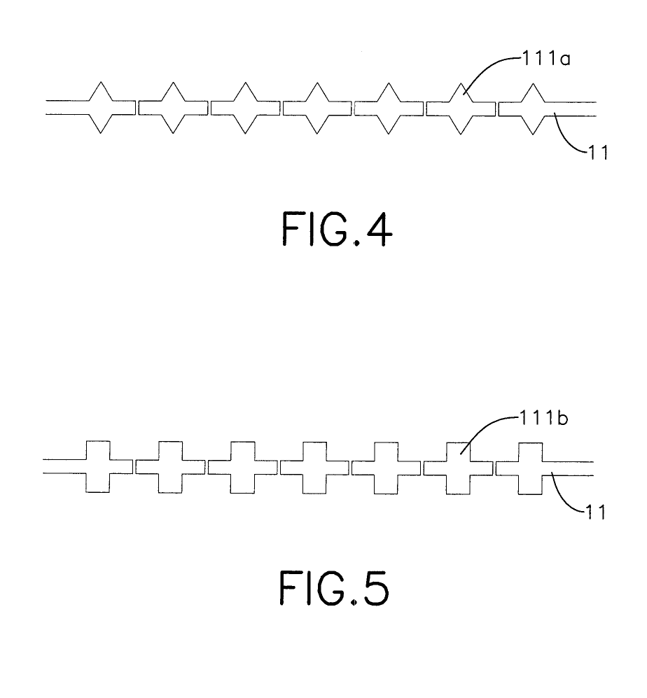

With reference to FIGS. 1, 4 and 5, a dental towel (10) in accordance with the present invention may be rectangular or polygonal shaped and has at least four edges, a straight tear line (11), a surrounding band (15), a curved tear line (12) and a removable section (17).

One of the edges is an oriented edge (13) that is oriented toward a patient.

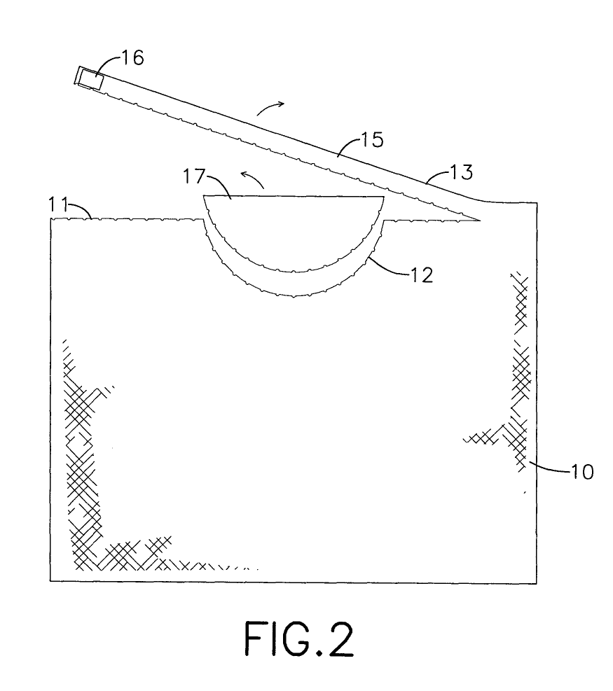

With further reference to FIG. 2, the straight tear line (11) parallels the oriented edges (13), is near one of the oriented edge (13) and has two ends and multiple hollow nodes (111, 111a, 111b). A length of the straight tear line (11) is shorter than a length of the oriented edge (13). One of the ends of the straight tear line (11) is formed on one of an adjacent edge (14) that is adjacent to the oriented edge (13). The hollow nodes (111, 111a, 111b) are formed along the straight tear line (11) at intervals and each node (111, 111a, 111b). A shape of the hollow nodes (111, 111a, 111b) may be circular, triangular, rectangular or polygonal. A user can tear the dental towel (10) from the end of the straight tear line (11) on the adjacent edge (14) and along the straight tear line (11).

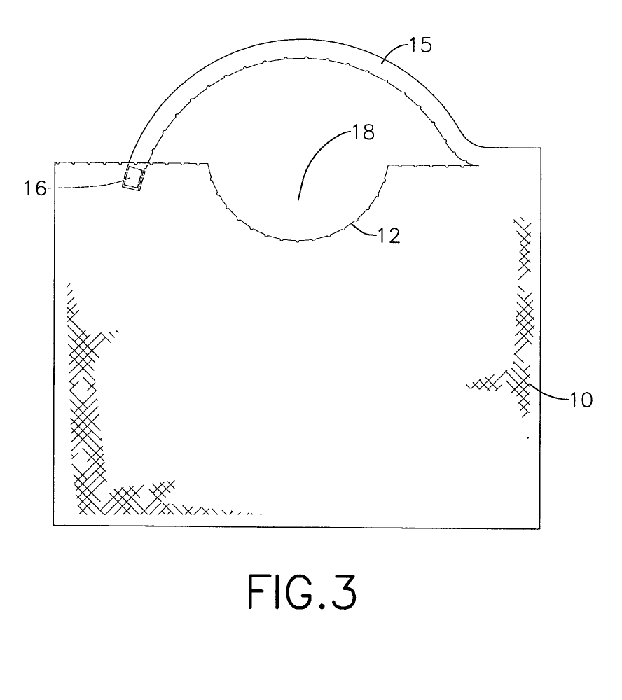

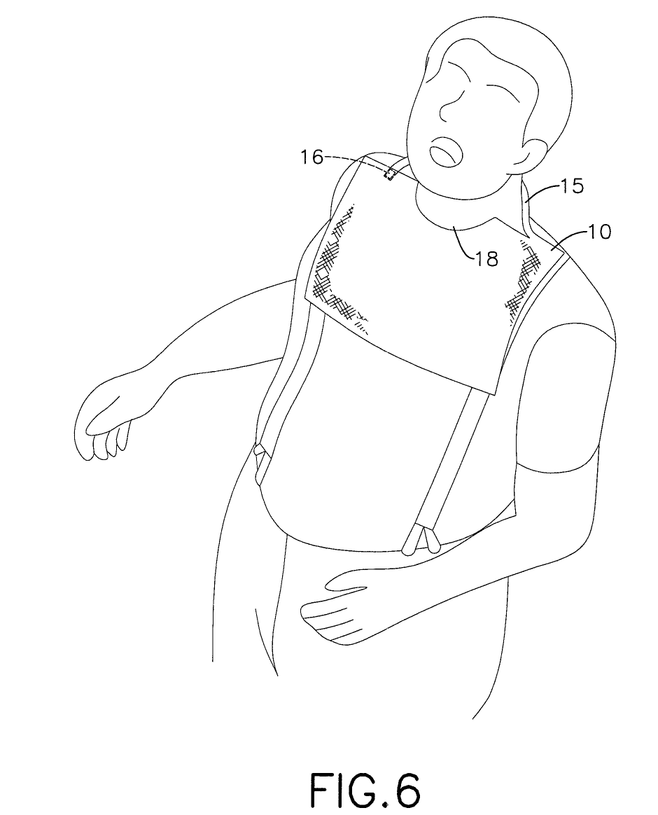

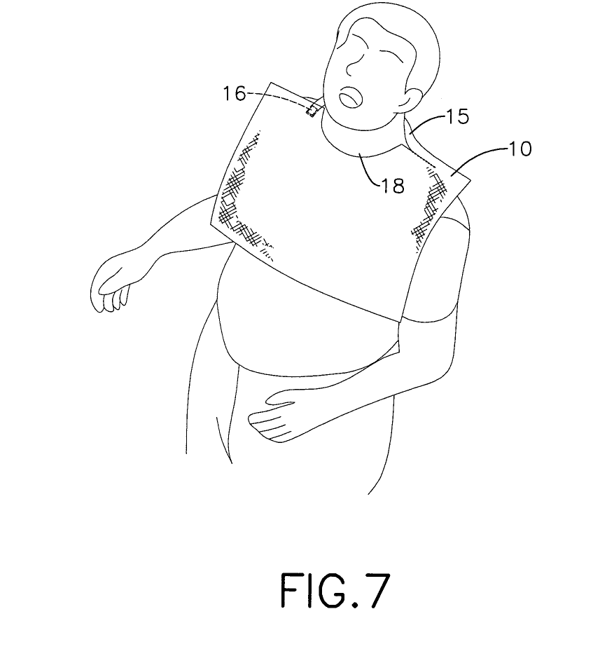

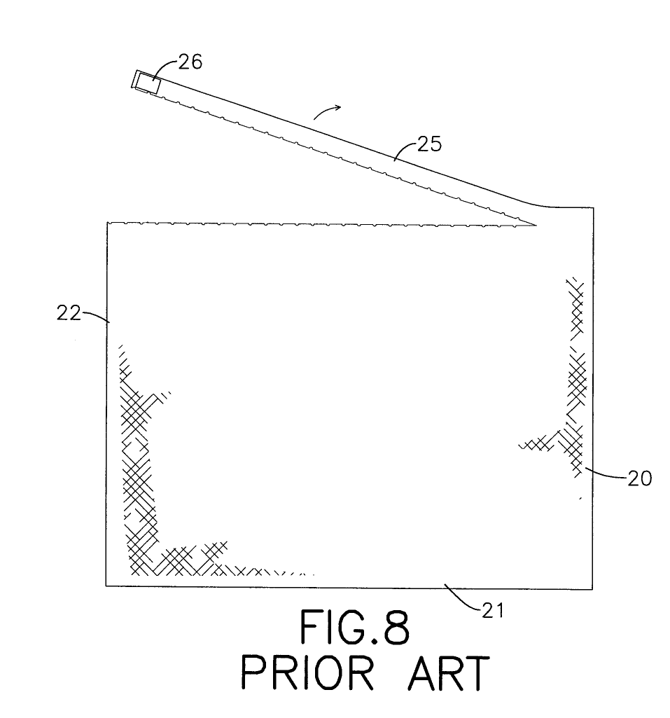

With further reference to FIGS. 3 and 6, the surrounding band (15) is mounted between the straight tear line (11) and the oriented edge (13), is used to surround patient's neck and has two opposite surfaces, an end and an adhesive member (16). The adhesive member (16) is mounted on one of the surfaces and near the end of the surrounding band (15) to stick on the dental towel (10) after surrounding the patient's neck without using a line with clips.

The curved tear line (12) is formed in the dental towel (10) and has two ends. Each end communicates with the straight tear line (11).

The removable section (17) is formed between the straight tear line (111, 111a, 111b) and the curved tear line (12), may be semicircular and can be removed when the user tears the dental towel (10) along the straight tear line (111, 111a, 111b) and the curved tear line (12) to allow the dental towel (10) to form a gap (18). The gap (18) may be semicircular and will not against the patient's neck.

The user can first tear easily the dental towel (10) from the end of the straight tear line (11) on the adjacent edge (14) and along the straight tear line (11) and stop tearing at one of the hollow node (111, 111a, 111b) according to a dimension of the patient's neck. The user than tears the dental towel (10) along the curved tear line (12) and removes the removable section (17) to form the gap (18). The surrounding band (12) and the gap (18) surround the patient's neck and the adhesive member (16) are convenient for the user to stick on the dental towel and to position the dental towel (10) in front of patient's chest. The gap (18) prevents the patient's neck from being against the dental towel (10), so the patient will feel comfortable.

With further reference to FIG. 8, no matter the patients are adults or children, males or females, the dental towel (10) can be torn to obtain the surrounding band (15) with a desired length to fit patient's neck. Thus, the dental towel (10) can be positioned in front of patient's chest. Thus, water or other agents will not spray to contaminate patient's clothes and neck.

Even though numerous characteristics and advantages of the present invention have been set forth in the foregoing description, together with details of the structure and function of the invention, the disclosure is illustrative only. Changes may be made in detail, especially in matters of shape, size and arrangement of parts within the principles of the invention to the full extent indicated by the broad general meaning of the terms in which the appended claims are expressed.

What is claimed is:

1. A dental towel comprising:
at least four edges and one of the edges being oriented edge;
a straight tear line paralleling the oriented edge, being near one of the oriented edge and having
a length being shorter than a length of the oriented edges;
two ends and one of the ends formed on an adjacent edge that is adjacent to the oriented edge; and
multiple hollow nodes formed along the straight tear line at intervals; and
a surrounding band mounted between the straight tear line and the oriented edge and having an adhesive member mounted on the surrounding band to stick on the dental towel when the dental towel is used.

2. The dental towel as claimed in claim 1, wherein the surrounding band further has
two opposite surfaces; and
an end; and
the adhesive member is mounted on one of the surfaces and near the end of the surrounding band.

3. The dental towel as claimed in claim 1, wherein each hollow node is circular.

4. The dental towel as claimed in claim 1, wherein each hollow node is triangular.

5. The dental towel as claimed in claim 1, wherein each hollow node is rectangular.

6. The dental towel as claimed in claim 1, wherein each hollow node is polygonal.

7. A dental towel and comprising:
at least four edges and one of the edges being oriented edge;
a straight tear line paralleling the oriented edge, being near one of the oriented edge and having
a length being shorter than a length of the oriented edges;
two ends and one of the ends formed on an adjacent edge that is adjacent to the oriented edge; and
multiple hollow nodes formed along the straight tear line at intervals; and
a surrounding band mounted between the straight tear line and the oriented edge and having an adhesive member mounted on the surrounding band to stick on the dental towel when the dental towel is used.
a curved tear line formed in the dental towel and having two ends and each end communicating with the straight tear line; and
a removable section formed between the straight torn line and the curved tear line and removed when the dental towel is used to form a gap.

8. The dental towel as claimed in claim 7, wherein the surrounding band further has
two opposite surfaces; and
an end; and
the adhesive member is mounted on one of the surfaces and near the end of the surrounding band.

9. The dental towel as claimed in claim 7, wherein the removable section is semicircular to form the gap being semicircular.

10. The dental towel as claimed in claim 7, wherein each hollow node is circular.

11. The dental towel as claimed in claim 7, wherein each hollow node is triangular.

12. The dental towel as claimed in claim 7, wherein each hollow node is rectangular.

13. The dental towel as claimed in claim 7, wherein each hollow node is polygonal.
